(19) United States
(12) Patent Application Publication
(10) Pub. No.: US 2019/0059847 A1
(43) Pub. Date: Feb. 28, 2019

(54) Glow in the Dark Stethoscope and Method of Manufacturing the Same

(71) Applicant: Medline Industries, Inc, Northfield, IL (US)

(72) Inventors: Molly Moon, Chicago, IL (US); Ben Duck, McHenry, IL (US)

(21) Appl. No.: 15/687,070

(22) Filed: Aug. 25, 2017

(51) Int. Cl. A61B 7/02

(52) U.S. Cl. CPC A61B 7/02

(57) ABSTRACT

A stethoscope (100) includes one or more earpieces (101,102), a sound receiving bell (104), and at least one acoustic conduit (103) coupling the one or more earpieces and the sound receiving bell. A photoluminsecent pigmented material (115) is applied to the at least one acoustic conduit to make it glow in the dark.

### BACKGROUND

### Technical Field

This disclosure relates generally to devices, and more particularly to medical devices.

### Background Art

Medical professionals employ diagnostic tools to determine the state of a patient's health. For example, blood pressure cuffs are used to attain a patient's systolic and diastolic blood pressure. Thermometers are used to determine a patient's temperature. Syringes are used to draw samples of a patient's blood.

One iconic diagnostic tool is the stethoscope. Stethoscopes are aural devices with which a medical professional can evaluate cardiopulmonary conditions, including heart rate, respiratory conditions, and intestinal conditions. Stethoscopes generally include a “bell,” which functions as a sound receiver. The bell is coupled to acoustic tubing, which is in turn coupled to earpieces.

Patients with experience do not fear a stethoscope, understanding it to be a painless and simple external tool for the medical professional to use. However, children are easily frightened by the long, looping tools and cold, shiny, metal bell. It would be advantageous to have an improved stethoscope that was not only less frightening to children, but suitable for use in a wide range of conditions.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to further illustrate various embodiments and to explain various principles and advantages all in accordance with the present disclosure.

FIG. 1 illustrates one explanatory stethoscope in accordance with one or more embodiments of the disclosure.

FIG. 2 illustrates one explanatory stethoscope glowing in the dark in accordance with one or more embodiments of the disclosure.

FIG. 3 illustrates a medical professional using one explanatory stethoscope, to the pleasure and delight of a young patient, in accordance with one or more embodiments of the disclosure.

FIGS. 4-5 illustrate one explanatory method of manufacturing stethoscope tubing in accordance with one or more embodiments of the disclosure.

FIG. 6 illustrates another explanatory stethoscope, along with one or more method steps, in accordance with one or more embodiments of the disclosure.

FIG. 7 illustrates another explanatory stethoscope, along with one or more method steps, in accordance with one or more embodiments of the disclosure.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the present disclosure.

### DETAILED DESCRIPTION OF THE DRAWINGS

Embodiments of the disclosure are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, and the like may be used solely to distinguish one entity or action from another entity or action without necessarily requiring or implying any actual such relationship or order between such entities or actions.

As used herein, components may be “operatively coupled” when information can be sent between such components, even though there may be one or more intermediate or intervening components between, or along the connection path. The terms “substantially” and “about” are used to refer to dimensions, orientations, or alignments inclusive of manufacturing tolerances. Thus, a “substantially orthogonal” angle with a manufacturing tolerance of plus or minus two degrees would include all angles between 88 and 92, inclusive. Also, reference designators shown herein in parenthesis indicate components shown in a figure other than the one in discussion. For example, talking about a device (10) while discussing figure A would refer to an element, 10, shown in figure other than figure A.

Embodiments of the disclosure contemplate that children can be frightened by medical devices. When a child is in a medical facility, such as a hospital, it is scary enough. The fright can be compounded, however, when a doctor or other medical professional begins examining the child with medical instruments, which frequently include shiny, cold, stainless steel components. Even the innocuous stethoscope can be frightening to a child that has not had experience being examined.

This fear can be compounded when the stethoscope is introduced in an unusual environment. For example, if a child is spending the night in a hospital, nurses or doctors may routinely check in to ascertain the child's physical condition. If the light is suddenly turned ON, this may startle a child. The fright is exacerbated when a medical device is thrust forward toward the child. Embodiments of the disclosure contemplate that it would be preferable to examine the child without turning ON the light in some cases. However, this is hard to do without further frightening the child. Moreover, the medical professional may not be able to sufficiently see in a darkened room to make the diagnosis.

Embodiments of the disclosure advantageously solve both issues by providing a glow in the dark stethoscope. In one or more embodiments, a stethoscope comprises one or more earpieces, a sound-receiving bell, and at least one acoustic conduit coupling the one or more earpieces to the sound-receiving bell. In one embodiment, one acoustic conduit couples the one or more earpieces to the sound-receiving bell. In another embodiments, two acoustic conduits couple the one or more earpieces to the sound-receiving bell, with a first acoustic conduit coupling a first earpiece to the sound-receiving bell, and a second acoustic conduit coupling a second earpiece to the sound-receiving bell. Other stethoscope configurations will be obvious to those of ordinary skill in the art having the benefit of this disclosure.

In one or more embodiments, a photoluminescent pigmented material is applied to the acoustic conduit. In one embodiment, the photoluminescent pigmented material comprises europium doped strontium aluminate, and has the chemical formula SrAl.sub.2O.sub.4:Eu.sup+2,Dy.sup.+3. While this photoluminescent pigmented material works well in practice, other photoluminescent pigmented materials will be obvious to those of ordinary skill in the art having the benefit of this disclosure.

The application of the photoluminescent pigmented material to the acoustic tubing advantageously allows the acoustic tubing to glow in the dark. This provides a dual benefit. First, the fact that the photoluminescent pigmented material stores, and then releases, energy as light allows the medical professional to use the acoustic tubing as an illumination device to more readily make diagnostic ascertainments in low-light environments. Continuing the example from above, when entering the child's room at night, the medical professional may not even need to turn on the light. Instead, the medical professional can rely upon the photoluminescent pigmented material as an illumination device.

Advantageously, the use of the photoluminescent pigmented material offers advantages over prior art systems. Specifically, no power source or electrical wiring is required. This not only makes stethoscopes configured in accordance with embodiments of the disclosure lighter and easier to use, but also reduced the cost and reduces any maintenance requirement associated with having a stethoscope with phosphorescent properties.

Second, the fact that the acoustic tubing glows in the dark makes an otherwise frightening and scary procedure fun, exciting, and even delightful for the child. A child that may otherwise be scared by a shadowy figure now finds a friend in a doctor that seems to have the powers of a super hero due to the bright glow of light emitted by the photoluminescent pigmented material. This improves the overall healthcare experience. Not only for the child, but for the child's parents as well.

In sum, embodiments of the disclosure provide a stethoscope that includes one or more earpieces, a sound-receiving bell, and acoustic conduit that glows in the dark. The glow in the dark acoustic conduit couples the one or more earpieces to the sound-receiving bell. The use of glow in the dark acoustic conduit offers properties to the stethoscope that make it more fun, more interesting, and generally more exciting to patients of all ages. This is especially true with the patient is younger in age. It should be noted that while the medical device used for illustration in this disclosure is a stethoscope, photoluminescent pigmented materials in accordance with embodiments of the disclosure could be applied to other devices as well, including blood pressure cuffs, suction tubing, rubber tubing used as a tourniquet when drawing blood samples, and other medical and non-medical devices.

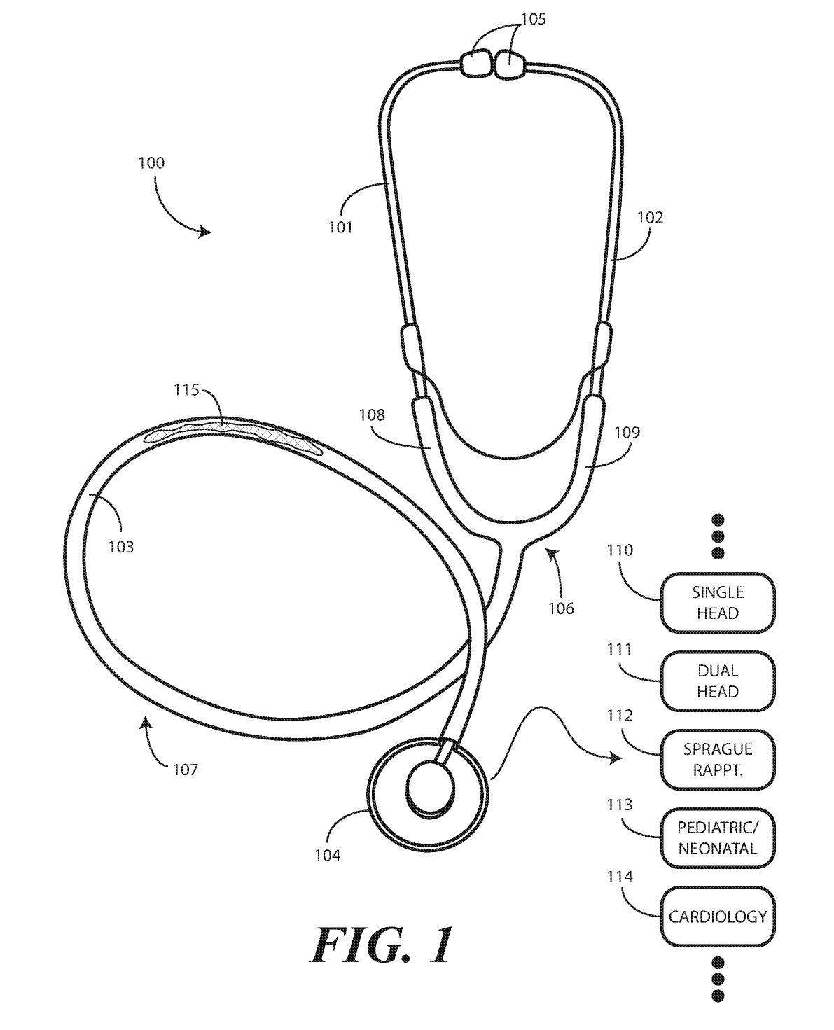

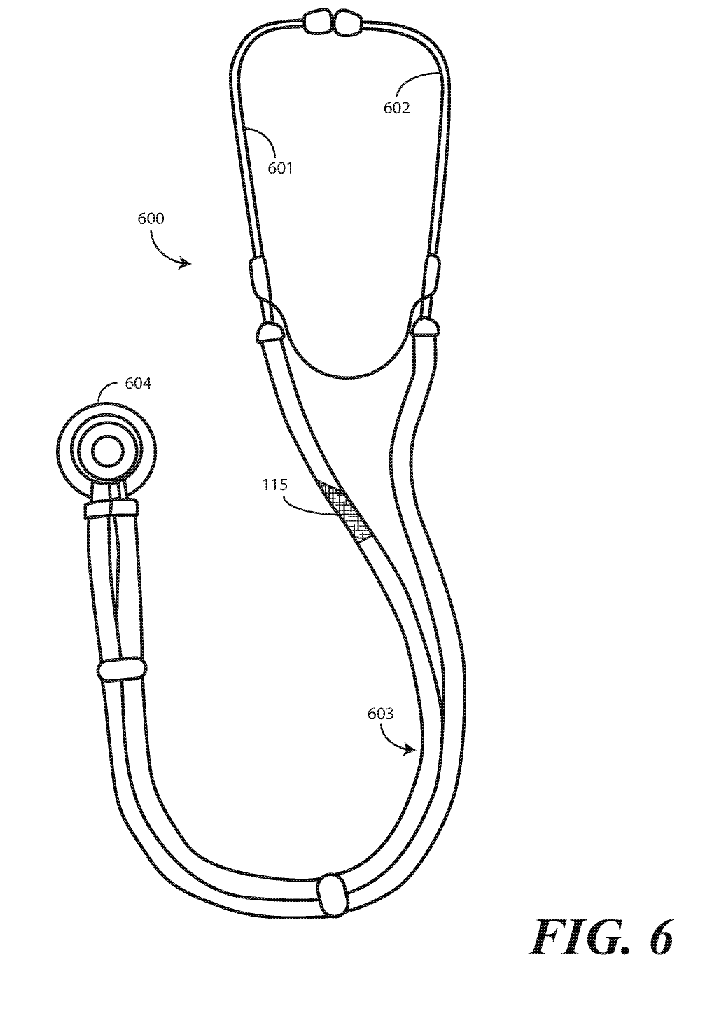

Turning now to FIG. 1, illustrated therein is one explanatory stethoscope 100 configured in accordance with one or more embodiments of the disclosure. The stethoscope 100 includes one or more earpieces 101,102, a sound-receiving bell 104, and at least one acoustic conduit 103 coupling the one or more earpieces 101,102 and the sound-receiving bell 104. In the illustrative embodiment of FIG. 1, the at least one acoustic conduit 103 comprises a single acoustic conduit. However, in other embodiments such as that shown in FIG. 6 below, in other embodiments two acoustic conduits can be used. Other configurations will be obvious to those of ordinary skill in the art having the benefit of this disclosure.

In one embodiment, the at least one acoustic conduit 103 comprises acoustic tubing. In one embodiment, the acoustic tubing comprises vinyl tubing. In one or more embodiments, the acoustic tubing—prior to the application of any other material—comprises optically transparent or translucent vinyl tubing. Other types of tubing can be used as the acoustic conduit 103 as well, as will be obvious to those of ordinary skill in the art having the benefit of this disclosure.

In one or more embodiments, the at least one acoustic conduit 103 couples the one or more earpieces 101,102, which are sometimes referred to as “binaurals” when used in pairs. Plastic ear cushions 105 can be coupled to the ends of the one or more earpieces 101,102 to make insertion thereof more comfortable in the ear.

In one or more embodiments, the at least one acoustic conduit 103 comprises a mechanical (non-electric) sound-wave conducting conduit that extends between twelve and twenty four inches from the one or more earpieces 101,102 to the sound-receiving bell 104. The illustrative acoustic conduit 103 of FIG. 1 includes a Y-shaped region 106 and an extension region 107. The Y-shaped region 106 defines a first upper extension 108 that couples to a first earpiece 101 and a second upper extension 109 that couples to a second earpiece 102. The extension region 107 extends distally downward from the base of the Y-shaped region 106 to the sound-receiving bell 104. It should be noted that the at least one acoustic conduit 103 can be replaced with other sound-transmitting devices. Such sound-transmitting devices may be mechanical acoustic wave-guides, or may be electrical signal conduits in other embodiments. Still other configurations will be obvious to those of ordinary skill in the art having the benefit of this disclosure.

The sound-receiving bell 104 is placed against the chest (or other part) of a patient and collects acoustic signals emanating from the patient. The sound-receiving bell 104 can take any of a variety of forms. In one embodiment, the sound-receiving bell 104 is a single-head bell 110. In another embodiment, the sound-receiving bell 104 is a dual-head bell 111. In still another embodiment, the sound-receiving bell 104 is a Sprague-Rappaport bell 112. In yet another embodiment, the sound-receiving bell 104 is a pediatric or neonatal bell 113. In still another embodiment, the sound-receiving bell 104 is a cardiology bell 114, which is a deeper bell than the other bells, having more of a cone shape than a parabolic or circular shape. Other types of sound-receiving bells will be obvious to those of ordinary skill in the art having the benefit of this disclosure.

In one or more embodiments, a photoluminescent pigmented material 115 is applied to the acoustic conduit 103. The photoluminescent pigmented material 115 radiates visible light after storing energy by being exposed to electromagnetic energy for a period of time. In one embodiment, the photoluminescent pigmented material 115 is applied to an exterior of the at least one acoustic conduit 103, as illustrated in FIG. 1. In another embodiment, the photoluminescent pigmented material 115 is applied to the interior of the at least one acoustic conduit 103. Recall from above that in one or more embodiments the acoustic conduit 103 comprises an optically transparent or translucent vinyl tubing. Advantageously, applying the photoluminescent pigmented material 115 to the interior of the acoustic conduit 103 prevents the same from being damaged by objects coming into contact with the acoustic conduit 103. In still other embodiments, the photoluminescent pigmented material 115 is integrated into the material of the acoustic conduit 103 itself. For example, the photoluminescent pigmented material 115 can be mixed into the vinyl when the acoustic conduit 103 is being extruded, and so forth. Other ways of integrating the photoluminescent pigmented material 115 into, or applying the photoluminescent pigmented material 115 to, the acoustic conduit 103 will be obvious to those of ordinary skill in the art having the benefit of this disclosure.

In one or more embodiments, the photoluminescent pigmented material 115 comprises europium doped strontium aluminate. For example, the photoluminescent pigmented material 115 can be an inorganic admixture of strontium, aluminum, and oxygen, which is mixed with a combination of europium oxide (Eu.sub.2O.sub.3) and dysprosium oxide (Dy.sub.2O.sub.3). In one or more embodiments the photoluminescent pigmented material 115 has the chemical formula SrAl.sub.2O.sub.4:Eu.sup+2,Dy.sup.+3.

As used herein, an “admixture” is a mixture where one constituent dominates, with other constituents being included in minor proportions. Accordingly, in one or more embodiments the photoluminescent pigmented material 115 comprises at least ninety-eight percent dialuminum strontium tetraoxide by weight. In one or more embodiments, the photoluminescent pigmented material 115 comprises less than one percent europium oxide by weight. In one or more embodiments, the photoluminescent pigmented material 115 comprises less than one percent dysprosium oxide by weight.

While europium doped strontium aluminate is one suitable photoluminescent pigmented material 115, others will be obvious to those of ordinary skill in the art having the benefit of this disclosure. For example, in another embodiment the photoluminescent pigmented material 115 comprises Zinc Sulfide. In another embodiment, the photoluminescent pigmented material 115 comprises Strontium Aluminate. Of course, combinations of these photoluminescent pigmented materials can be used as well. Still other examples of photoluminescent pigmented materials will be obvious to those of ordinary skill in the art having the benefit of this disclosure.

In one or more embodiments, the photoluminescent pigmented material 115 has a yellow-green color. As used herein, “yellow-green” refers to a color that is a mixture of about fifty-percent light that stimulates the yellow-green cone receptors of the eye. Illustrating by example, in one or more embodiments the photoluminescent pigmented material 115 has substantially the same color that is represented in electrical systems using hex triplet #7FFF00, or alternatively using standard red, green, and blue (sRGB.sup.B) values of 127,255,0, or alternatively using standard cyan, magenta, yellow key (CMYK.sup.H) values of 50,0,100,0, or alternatively hue, saturation, and value (HSV) representations of 90 degrees, one-hundred percent, one-hundred percent. However, other variations of yellow-green can be used as well.

Illustrating by example, in one embodiment the photoluminescent pigmented material 115 has substantially the same color as that represented using hex triplet #DFFF00, sRGB.sup.B values of 233,255,0, CMYK.sup.H values of 13,0,100,0, or HSV representations of 68 degrees, one-hundred percent, one-hundred percent. In another embodiment the photoluminescent pigmented material 115 has substantially the same color as that represented using hex triplet #E3FF00, sRGB.sup.B values of 227,255,0, CMYK.sup.H values of 11,0,100,0, or HSV representations of 67 degrees, one-hundred percent, one-hundred percent. In another embodiment the photoluminescent pigmented material 115 has substantially the same color as that represented using hex triplet #ADFF2F, sRGB.sup.B values of 173,255,0, CMYK.sup.H values of 32,0,82,0, or HSV representations of 83 degrees, eighty-one percent, one-hundred percent. In another embodiment the photoluminescent pigmented material 115 has substantially the same color as that represented using hex triplet #9ACD32, sRGB.sup.B values of 154,205,50, or HSV representations of 90 degrees, sixty percent, fifty-four percent. Other examples of yellow-green will be obvious those of ordinary skill in the art having the benefit of this disclosure.

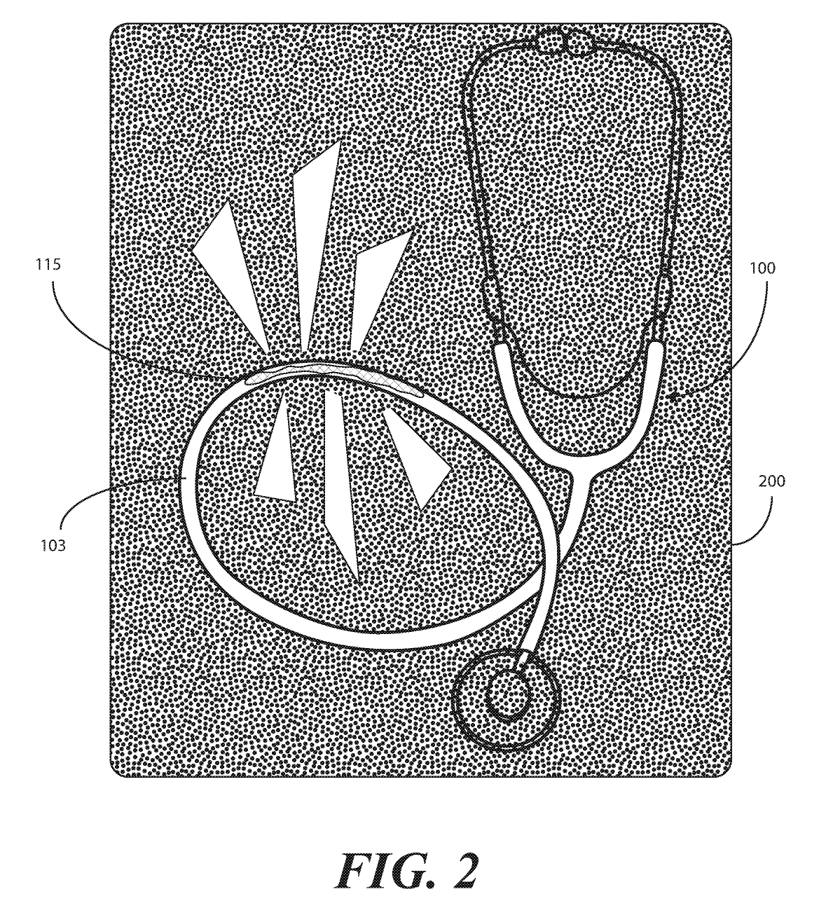

The application of the photoluminescent pigmented material 115 to the acoustic conduit 103 advantageously makes the acoustic conduit 103 glow in the dark. Turning now to FIG. 2 illustrated therein is the stethoscope 100 of FIG. 1, placed into a dark environment 200, after the photoluminescent pigmented material 115 has been charged. As shown, the acoustic conduit 103 glows in the dark. This effect can bedazzle patients old and young alike. However, the impact made by the acoustic conduit 103 glowing the dark is especially impressive to younger patients.

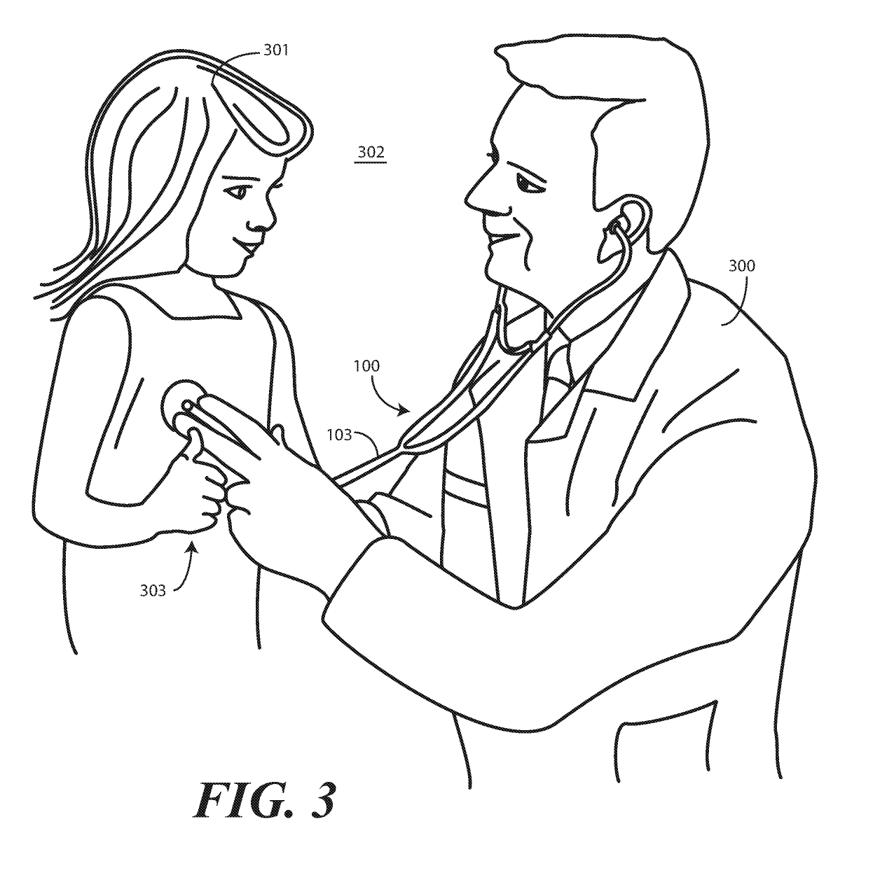

Turning now to FIG. 3, illustrated therein is a medical professional 300 treating a young patient 301 using a stethoscope configured in accordance with one or more embodiments of the disclosure. As shown the acoustic conduit 103 is glowing brightly due to the fact that the environment 302 is a low-light environment and the photoluminescent pigmented material (115) is photoluminescing. The young patient 301 is thrilled and delighted, as is evidenced by her thumbs-up gesture 303.

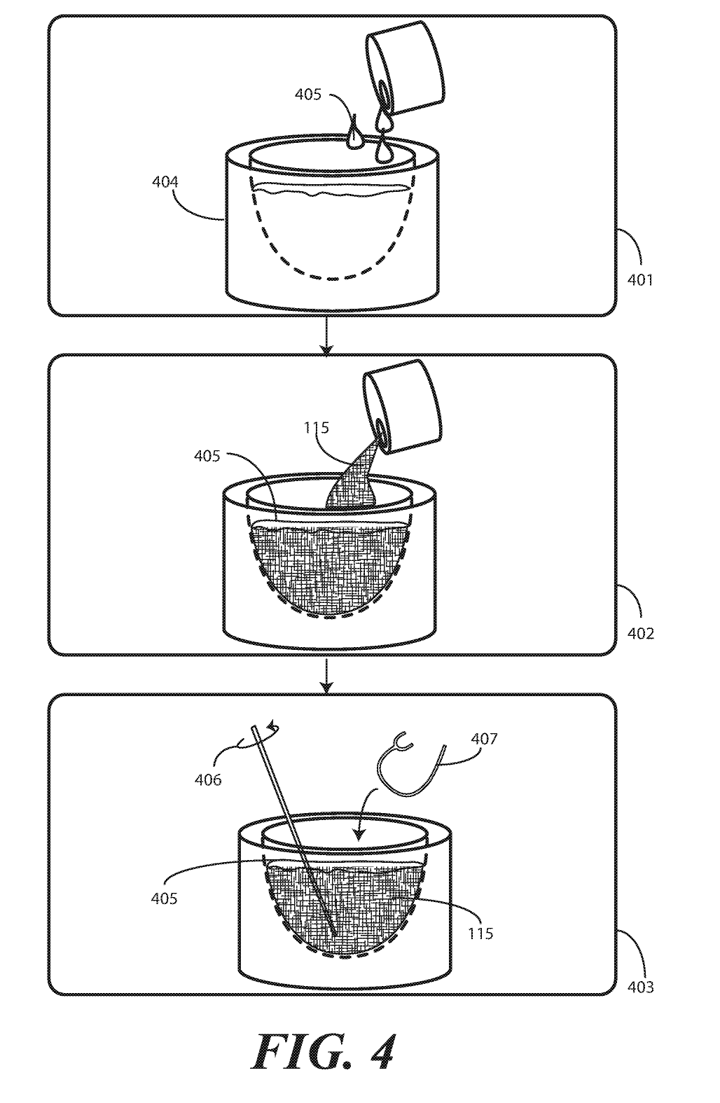

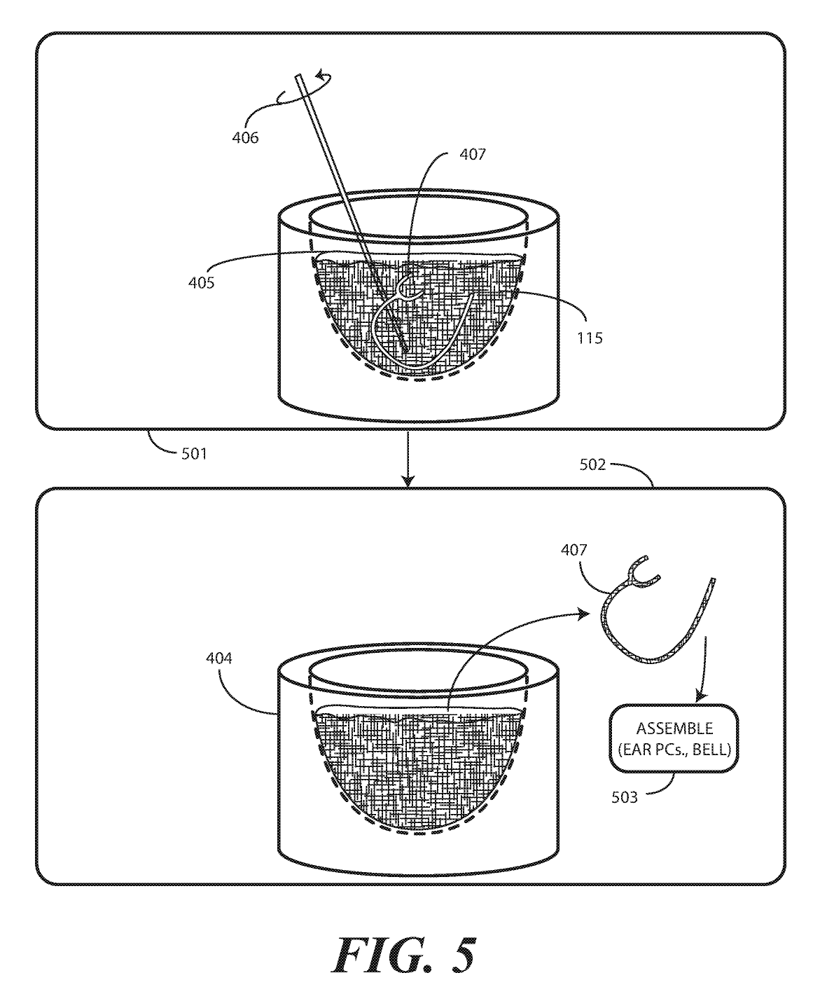

Turning now to FIGS. 4-5 illustrated therein is one explanatory method for manufacturing a stethoscope in accordance with one or more embodiments of the disclosure. Beginning at step 401, a vat 404 is filled with a solvent 405. In one embodiment, the solvent 405 is water. In another embodiment, the solvent 405 comprises nitrocellulose. Other solvents will be obvious to those of ordinary skill in the art having the benefit of this disclosure.

At step 402, the photoluminescent pigmented material 115 is added to the solvent 405. In one embodiment, the ratio of photoluminescent pigmented material 115 to solvent is about two ounces of the photoluminescent pigmented material 115 per gallon of the solvent 405. In one or more embodiments, the photoluminescent pigmented material 115 is originally in a solid state, but dissolves, partially dissolves, or disperses in the solvent 405. At step 403, the solvent 405, with the photoluminescent pigmented material 115 dispersed therein, is stirred 406. Acoustic tubing 407 is then placed within the solvent 405.

Turning now to FIG. 5, at step 501, the solvent 405 is stirred 406 while the acoustic tubing 407 is submerged within the solvent 405. In one embodiment, the solvent 405 is continuously stirred 406 when the acoustic tubing 407 is submerged within the solvent due to the fact that the photoluminescent pigmented material 115 is insoluble in organic solvents. The continuous stirring 406 prevents the photoluminescent pigmented material 115 from settling to the bottom, and instead allows the photoluminescent pigmented material 115 to be applied to the acoustic tubing 407. At step 501, the acoustic tubing 407is removed from the vat 404 and is attached 503 to one or more earpieces (101,102) and a sound-receiving bell (104) as previously described. The acoustic tubing 407 may be passed through a drying process (not shown) such as passing the acoustic tubing 407 through a heat tunnel or under a dryer.

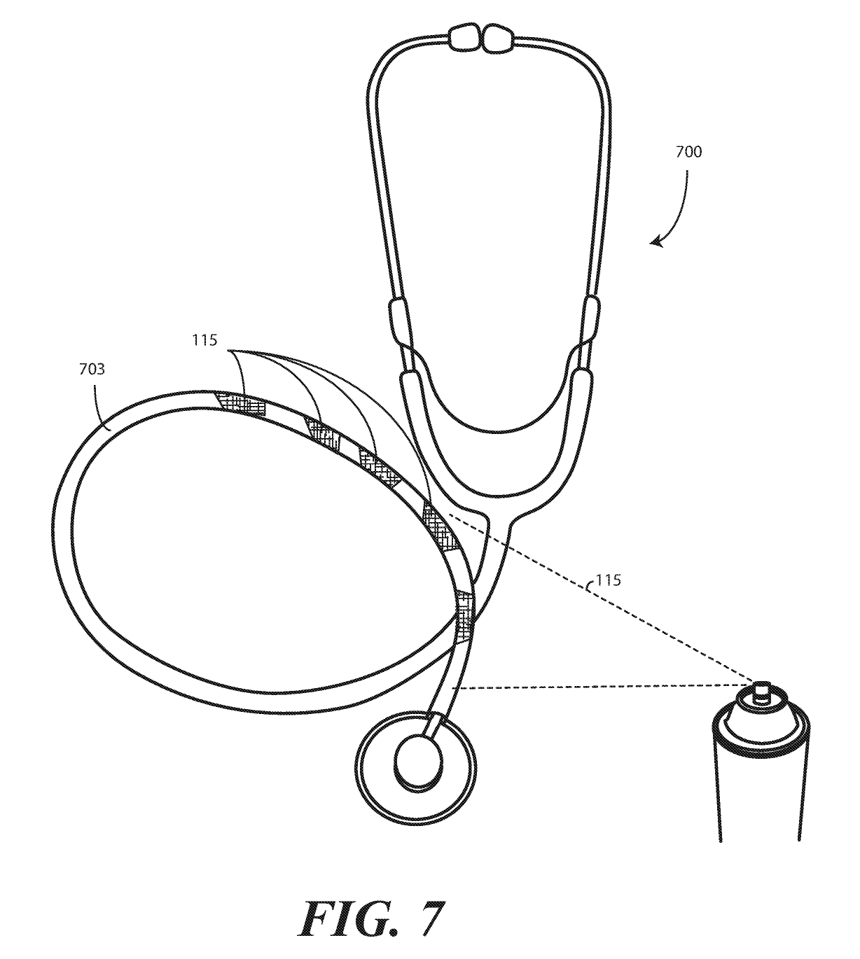

It should be noted that both stethoscopes, and the methods for manufacturing the same, can be varied from the devices and methods previously described. Turning now to FIGS. 6 and 7, illustrated therein are a few examples of how this can occur. Note that while these variations are shown only in FIGS. 6 and 7, the methods therein could be applied to previously described devices. Similarly, the devices therein could be manufactured using previously described methods, and so forth.

As shown in FIG. 6, a stethoscope comprises one or more earpieces 601,602 and a sound-receiving bell 604. As before, an acoustic conduit 603 that glows in the dark couples the one or more earpieces 601,602 to the sound-receiving bell 604. As previously described, in this embodiment the acoustic conduit 603 comprises transparent vinyl tubing. However, rather than being a single acoustic conduit, the acoustic conduit 603 of FIG. 6 comprises two acoustic conduits. A first acoustic conduit couples a first earpiece to the sound-receiving bell 604, while a second acoustic conduit couples a second earpiece to the sound-receiving bell 604.

As before, a photoluminescent pigmented material 115 is applied to the acoustic conduit 603. However, rather than using the method of FIGS. 4-5, in this illustrative embodiment the photoluminescent pigmented material 115 is integrated into the material of the acoustic conduit 603 itself. For example, the photoluminescent pigmented material 115 can be mixed into the vinyl when the acoustic conduit 603 is being extruded. Other ways of integrating the photoluminescent pigmented material 115 into, or applying the photoluminescent pigmented material 115 to, the acoustic conduit 603 will be obvious to those of ordinary skill in the art having the benefit of this disclosure.

Turning now to FIG. 7, in this embodiment another stethoscope 700 is shown. As bevore, a photoluminescent material 115 is applied to the acoustic conduit 703. However, rather than using the method of FIGS. 4-5, or integrated into the acoustic conduit 703 as in FIG. 6, in this illustrative embodiment the photoluminescent pigmented material 115 is sprayed upon the acoustic conduit 703. Additionally, rather than covering the entirety of the acoustic conduit 703, as was the case in FIG. 1, in FIG. 7 the photoluminescent pigmented material 115 is only selectively applied to portions of the acoustic conduit 703.

The locations at which the photoluminescent pigmented material 115 is disposed can vary based upon application or desire. For example, the selective locations can define a fashion statement, unique design, artistic design, or provide an aesthetically pleasing appearance in one or more embodiments. In other embodiments, the selective locations can provide a visible indication that the stethoscope 700 is, in fact, a glow in the dark stethoscope. Moreover, the selective deposition of the photoluminescent pigmented material 115 allows different, contrasting colors of the photoluminescent pigmented material 115 to be applied in patterns. For example, stripes of green photoluminescent pigmented material 115 can be applied with intermittent stripes of yellow photoluminescent pigmented material 115 disposed therebetween, and so forth.

In the foregoing specification, specific embodiments of the present disclosure have been described. However, one of ordinary skill in the art appreciates that various modifications and changes can be made without departing from the scope of the present disclosure as set forth in the claims below. Thus, while preferred embodiments of the disclosure have been illustrated and described, it is clear that the disclosure is not so limited. Numerous modifications, changes, variations, substitutions, and equivalents will occur to those skilled in the art without departing from the spirit and scope of the present disclosure as defined by the following claims. Accordingly, the specification and figures are to be regarded in an illustrative rather than a restrictive sense, and all such modifications are intended to be included within the scope of present disclosure. The benefits, advantages, solutions to problems, and any element(s) that may cause any benefit, advantage, or solution to occur or become more pronounced are not to be construed as a critical, required, or essential features or elements of any or all the claims.

1. A stethoscope, comprising:
one or more earpieces;
a sound receiving bell;
an acoustic conduit coupling the one or more earpieces and the sound receiving bell, the acoustic conduit comprising a Y-shaped region and an extension region; and
a photoluminescent pigmented material applied to a surface of the acoustic conduit so as to cover an entirety of the acoustic unit.

2. The stethoscope of claim 1, wherein the photoluminescent pigmented material is applied to an exterior of the acoustic conduit.

3. The stethoscope of claim 2, wherein the photoluminescent pigmented material comprises europium doped strontium aluminate.

4. The stethoscope of claim 2, wherein the photoluminescent pigmented material has the chemical formula SrAl.sub.2O.sub.4:Eu.sup+2,Dy.sup.+3.

5. The stethoscope of claim 2, wherein the photoluminescent pigmented material comprises an inorganic admixture.

6. The stethoscope of claim 5, wherein the inorganic admixture comprises at least ninety-eight percent dialuminum strontium tetraoxide by weight.

7. The stethoscope of claim 6, wherein the inorganic admixture comprises less than one percent europium oxide by weight.

8. The stethoscope of claim 6, wherein the inorganic admixture comprises less than one percent dysprosium oxide by weight.

9. The stethoscope of claim 6, wherein the acoustic conduit comprises a single acoustic conduit.

10. The stethoscope of claim 1, wherein the photoluminescent pigmented material is yellow-green in color.

11. The stethoscope of claim 1, wherein the acoustic conduit comprises vinyl tubing.

12. (canceled)

13. A method of manufacturing a stethoscope, the method comprising:
selectively applying a photoluminescent pigmented material only to portions of an acoustic tubing; and
attaching the acoustic tubing to one or more earpieces and a sound receiving bell.

14. The method of claim 13, wherein the applying comprises:
adding the photoluminescent pigmented material to a solvent; and
submerging the acoustic tubing in the solvent.

15. The method of claim 14, further comprising stirring the solvent while the acoustic tubing is submerged in the solvent.

16. The method of claim 15, wherein the stirring comprises continuously stirring the solvent while the acoustic tubing is submerged in the solvent.

17. The method of claim 13, wherein the applying comprises spraying the photoluminescent pigmented material on the acoustic tubing.

18. The method of claim 13, wherein the applying comprises selectively applying the photoluminescent material only to the portions of the acoustic tubing in a striped pattern.

19. A stethoscope, comprising:
one or more earpieces;
a sound receiving bell;
glow in the dark acoustic conduit coupling the one or more earpieces and the sound receiving bell.

20. The stethoscope of claim 19, wherein the glow in the dark acoustic conduit comprises:
transparent vinyl tubing; and
a photoluminescent pigmented material applied to an exterior of the transparent vinyl tubing.
